(19) United States
(12) Patent Application Publication
(10) Pub. No.: US 2010/0222402 A1
(43) Pub. Date: Sep. 2, 2010

(54) METHOD OF MANUFACTURING 4'-[[4-METHYL-6-(1-METHYL-1H-BENZIMIDAZOL-2-YL)-2-PROPYL-1H-BENZIMIDAZOL-1YL]METHYL]BIPHENYL-2-CARBOXYLIC ACID (TELMISARTAN)

(76) Inventors: Jan Stach, Praha (CZ); Stanislav Radl, Kvetnice (CZ); Josef Cinibulk, Praha (CZ); Ivo Strelec, Praha (CZ); Kamal Jarrah, Praha (CZ)

Correspondence Address: Pearl Cohen Zedek Latzer, LLP, 1500 Broadway, 12th Floor, New York, NY, 10036, US

(21) Appl. No.: 12/667,732

(22) PCT Filed: Jul. 8, 2008
(86) PCT No.: PCT/CZ08/00080

§ 371 Date: Apr. 29, 2010

(30) Foreign Application Priority Data
Jul. 9, 2007 (CZ) PV 2007-457

(51) Int. Cl. A61K 31/4184; C07D 403/10

(52) U.S. Cl. 514/394; 548/305.4

(57) ABSTRACT

A carboxylic acid of the general formula R¹COOH, wherein R¹ is the hydrogen atom or a C₁-C₄ alkyl, is added to a solution of the potassium salt of telmisartan in an alcohol of the formula R²OH with the water content lower than 2%, wherein R² is ethyl or methyl.

### TECHNICAL FIELD

The invention deals with an improved method of manufacturing 4′-[[4-methyl-6-(1-methyl-1H-benzimidazol-2-yl)-2-propyl-1H-benzimidazol-1-yl]methyl]biphenyl-2-carboxylic acid P (telmisartan) (I)

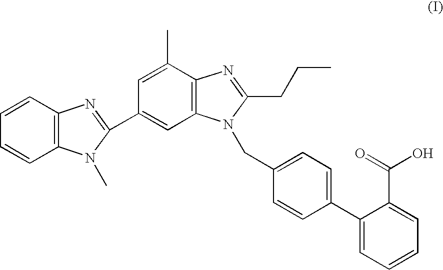

Telmisartan belongs to the group of angiotensin II antagonists, which are being therapeutically used as medicaments for the cardiovascular system, especially to control high blood pressure. A dosage form of telmisartan was introduced in the market in 1998 by Boehringer Ingelheim under the protected name Micardis^{R}. This group contains important drugs like losartan (Cozaar^{R}), irbesartan (Avapro^{R}), or valsartan (Diovan^{R}). However, unlike these substances telmisartan shows better efficiency even in the last hours of the administration interval.

### BACKGROUND ART

Telmisartan (I) is produced in accordance with the original patent of Boehringer Ingelheim (U.S. Pat. No. 5,591,762) from telmisartan text-butyl ester (II). The hydrolysis is carried out using of trifluoroacetic acid in the toxic solvent N,N-dimethylformamide.

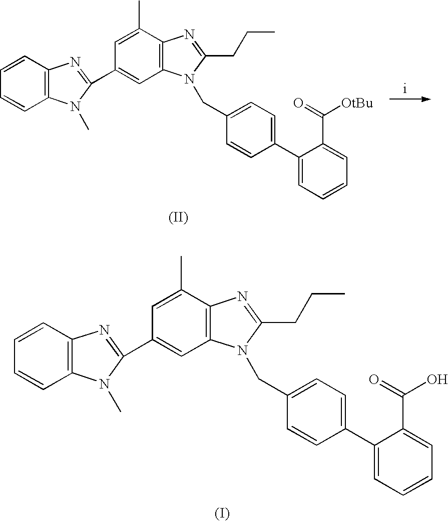

According to another patent applied by the same company (US 2004 236113) the manufacture was problematic and this is why this procedure was replaced with hydrolysis of the corresponding nitrile (III). However, during the hydrolysis, which is carried out with potassium hydroxide in ethylene glycol, a high temperature (160° C.) is used, which causes browning of the product, which must be subsequently purified by means of activated carbon. Also, the energy demands of several-ton production would be considerably high.

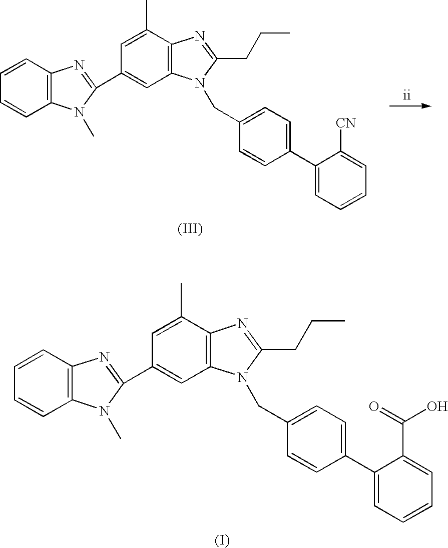

In a newer application of Cipla (WO 2005/10837) the last two synthetic steps (iii+iv) are combined and telmisartan is isolated after alkaline hydrolysis by acidifying of the reaction mixture in water or extraction with dichloromethane and precipitation with acetone. Both the ways of isolation are unsuitable for industrial production. In the case of telmisartan of crystalline form A its isolation from water or aqueous solutions of organic solvents is very difficult since a hardly filterable product is formed. Extraction of the product with dichloromethane and precipitation with acetone brings a well-filterable product, but the use of dichloromethane is virtually impossible from the point of view of environment protection.

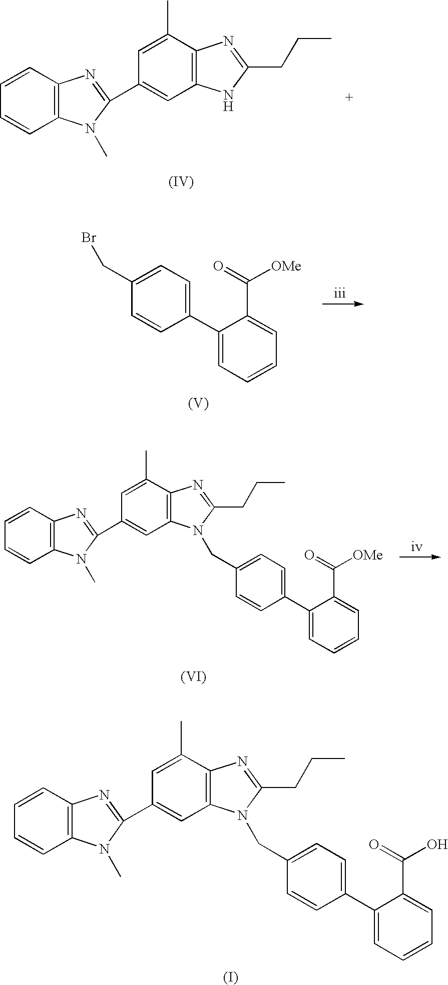

Another method has been described by Dr. Reddy (WO 2006/044754), which starts from telmisartan methylester hydrochloride, which is hydrolyzed to produce the potassium salt of termisartan, which is further acidified in aqueous acetonitrile; after isolation it crystallizes from a dichloromethane/methanol mixture and finally from methanol alone, and wherein a pressure apparatus is used for the dissolution in methanol at a temperature above its boiling point (80° C.). The result of this complex procedure, which manifests the already above mentioned shortcomings, is a low yield of the product.

The method of Teva (WO 2006/044648) is in many aspects similar to the above mentioned procedure of Cipla, wherein the last two steps of the synthesis are also combined. The method comprises phase separations, which lead to low yields (69%-80%) besides increased tediousness.

Matrix starts from telmisartan tert-butyl ester (II), which is first converted to telmisartan dihydrochloride, which in turn, by action of aqueous ammonia in methanol, provides telmisartan with a low total yield of 73%.

### DISCLOSURE OF INVENTION

The object of the invention is an improved method of manufacturing 4′-[[4-methyl-6-(1-methyl-1H-benzimidazol-2-yl)-2-propyl-1H-benzimidazol-yl]methyl]biphenyl-2-carboxylic acid (telmisartan) (I). The essence consists in the surprising finding that filterability of the crystalline form A of telmisartan in alcohols depends on the content of water and hence its isolation from an anhydrous solvent is best. Moreover, it has been surprisingly found out that filterability can be improved by the presence of potassium salts of carboxylic acids, which further significantly increases the yield of the process. In addition, the use of carboxylic acids for obtaining telmisartan from its potassium salt provides very good purity of the product.

A detailed description of the invention follows:

Boehringer Ingelheim have described, n U.S. Pat. No. 6,410,742, preparation of the new polymorph B, which is said to have better filterability than that of the originally described form A. However, the comparison experiment comprises crystallization from ethanol, wherein telmisartan is converted to an ammonium salt by means of aqueous ammonia and then telmisartan crystallizes by addition of acetic acid. Thus, during crystallization the system contains 2.5% of water, which substantially impairs filterability. In our case it has been surprisingly found out that the content of water in ethanol is of key importance for filterability of the reaction mixture. It has been established that filterability strongly depends on the content of water as well as inorganic salts. An experiment was performed wherein the time of filtration of the product was measured under the same conditions in dependence on the content of water and content of inorganic salts (the end of filtration is measured as disappearance of the solvent phase over aspirated crystals) (Table 1). Crystallization was carried out by addition of acetic acid or formic acid to an ethanolic or methanolic solution of the ammonium or potassium salt of telmisartan in accordance with U.S. Pat. No. 6,410,742.

| | | | Inorganic salt | | |
|---|---|---|---|---|---|
| Weight of | | Water | content (salt/ | Filtration time | |
| telmisartan | Solvent | content | telmisartan) | (minutes) | Yield |
| 10 g | ethanol | 2.5% (U.S. Pat. No. | 23% (ammonium | 10 | 90% |
| | | 6,410,742) | acetate) | | |
| 10 g | ethanol | 10% | 23% (ammonium | 155 | 91% |
| | | | acetate) | | |
| 10 g | ethanol | 1% | 23% (ammonium | 5 | 89% |
| | | | acetate) | | |
| 10 g | ethanol | 0.1% | 23% (ammonium | 2 | 87% |
| | | | acetate) | | |
| 10 g | methanol | 2.5% | 23% (ammonium | 11 | 91% |
| | | | acetate) | | |
| 10 g | methanol | 1% | 66% (potassium | 2 | 95% |
| | | | acetate) | | |
| 10 g | methanol | 1% | 90% (potassium | 2 | 97% |
| | | | acetate) | | |
| 10 g | methanol | 0.5% | 66% (potassium | 1 | 95% |
| | | | formate) | | |

The table shows that in the industrial scale the amount of water and inorganic salts will be the key parameter of the process. The amount of water has a principal impact on filterability of the product and an increased quantity of potassium salts of carboxylic acids reduces solubility of telmisartan and hence has a positive impact on the yield of the process. If the preparation of telmisartan starts from the corresponding methylester, it is also essential to get a product that does not contain inorganic substances. Therefore the inorganic salts used must display high solubility in the alcohols used.

In the course of experiments it has been found that the hydrolysis of telmisartan methylester can be most suitably carried out with potassium hydroxide in anhydrous methanol; after the reaction is complete, the crystalline form A of telmisartan is obtained by addition of acetic or formic acids. Although the product contains a considerable quantity of potassium acetate or formate, it has been found out that the reaction provides the product with a low content of potassium acetate or formate expressed by a low content of sulfate ash. Such mode of carrying out the reaction then complies with the requirements for a synthesis carried out in an industrial scale.

The invention will be elucidated in a more detailed way in the following examples. These examples, which illustrate the improvement of the procedure according to the invention, are of an illustrative character only and do not limit the scope of the invention in any aspect.

### EXAMPLES

### Example 1

### 4′-[[4-methyl-6-(1-methyl-1H-benzimidazol-2-yl)-2-propyl-1H-benzimidazol-1yl]methyl]biphenyl-2-carboxylic acid (telmisartan)

Telmisartan methylester (VI) (40 g) was refluxed in methanol (440 ml) with potassium hydroxide (14.9 g) for 24 hours. To the boiling solution, methanol (240 ml) and then acetic acid (45.5 g) were added. While boiling, the mixture was stirred for another 1 hour, after cooling to 4° C. the product was aspirated within 1 hour and washed with methanol (2×80 ml). After drying at the laboratory temperature (24 h) 35.18 g (90%) of the product were obtained.

Analytic assessment:

HPLC purity: 99.90%,

Content of residual solvents: methanol (below the detection limit)
acetic acid (360 ppm)

Titration content: 100.9%

Sulfate ash content: 0.04%

DSC: form A

### Example 2

### 4′-[[4-Methyl-6-(1-methyl-1H-benzimidazol-2-yl)-2-propyl-1H-benzimidazol-1yl]methyl]biphenyl-2-carboxylic acid (telmisartan)

Telmisartan methylester (VI) (20 g) was refluxed in methanol (300 ml) with potassium hydroxide (7 g) for 24 h. After addition of formic acid (17 g) and after cooling to 4° C. the product was aspirated within 1 hour and washed with methanol (2×80 ml). After drying at the laboratory temperature (24 h) 18.7 g (96%) of the product were obtained.

### Example 3

### 4′-[[4-methyl-6-(1-methyl-1H-benzimidazol-2-yl)-2-propyl-1H-benzimidazol-1yl]methyl]biphenyl-2-carboxylic acid (telmisartan)

Telmisartan methylester (VI) (20 kg) was refluxed in methanol (400 l) with potassium hydroxide (7 kg) for 24 h. After addition of acetic acid (20 kg) and cooling to 4° C. the product was aspirated within 1 hour and washed with methanol (2×80 l). After drying at the laboratory temperature (24 h) 18.5 kg (95%) of the product were obtained.

### Example 4

### 4′-[[4-methyl-6-(1-methyl-1H-benzimidazol-2-yl)-2-propyl-1H-benzimidazol-1yl]methyl]biphenyl-2-carboxylic acid (telmisartan)

Telmisartan methylester (40 g) was refluxed in methanol (240 ml) with potassium hydroxide (14.9 g) for 24 h. To the boiling solution methanol (240 ml) and then acetic acid (45.5 g) were added. After cooling to 4° C. the product was aspirated within 1 hour and washed with methanol (2×80 ml). After drying at the laboratory temperature (24 h) 36 g (92%) of the product were obtained.

1. A method of manufacturing the crystalline form A of telmisartan (I) 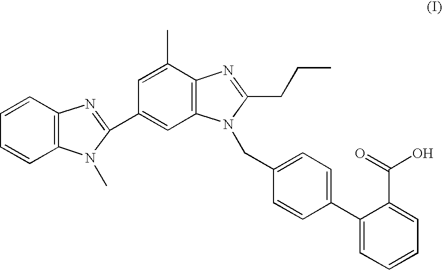
wherein a carboxylic acid of the general formula R¹COOH, in which R¹ is the hydrogen atom or a C₁-C₄ alkyl, is added to a solution of the potassium salt of telmisartan (VII) 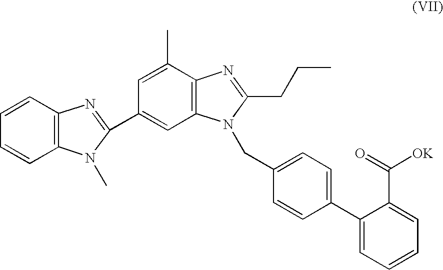
in an alcohol of the formula R²OH with the water content lower than 2%, wherein R² is ethyl or methyl.

2. The method according to claim 1, wherein the potassium salt of telmisartan is obtained by hydrolysis of a telmisartan alkyl ester of the general formula (VIII), 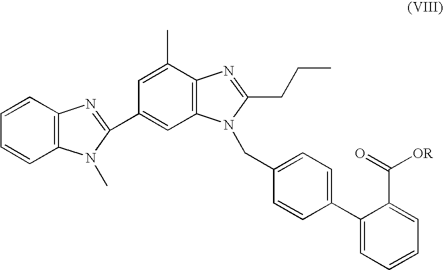
in which R is methyl or ethyl, with potassium hydroxide in an alcohol of the formula R²OH, wherein R² is ethyl or methyl.

3. The method according to claim 1, wherein the potassium salt of telmisartan is obtained by neutralization of telmisartan of formula (I) with potassium hydroxide,

4. The method according to claim 1, wherein the content of the obtained potassium salt of the carboxylic acid R¹COOH, expressed as the salt/telmisartan weight ratio, is 20%-150% during crystallization,

5. The method according to claim 1, wherein acetic acid or formic acid is used as the carboxylic acid.

6. The method according to claim 1, wherein the content of water in the system is lower than 1%.

7. A method of manufacturing the crystalline form A of telmisartan (I), wherein a telmisartan ester (VIII) is heated up in methanol with the water content lower than 1% by weight together with potassium hydroxide to the boiling temperature for 12 to 48 hours, formic or acetic acid is added to the solution and after cooling, the crystalline form A of telmisartan is separated.

8. The method according to claim 7, wherein telmisartan of form A is crystallized at a temperature of −10 to +10° C.

9. The method according to claim 7, wherein the ratio of telmisartan, potassium hydroxide and formic or acetic acid is selected so as to produce the resulting weight ratio of the potassium salt of the selected organic acid to the resulting telmisartan of 1:2 to 6:5

10. The crystalline product telmisartan of crystalline form A, obtainable by the method according to claim 9

11. Suspension of telmisartan of crystalline form A in methanol with water content of less than 1% by weight, obtainable by the method according to claim 9
